(19) United States
(12) Patent Application Publication
(10) Pub. No.: US 2021/0369802 A1
(43) Pub. Date: Dec. 2, 2021

(54) CANNABIS PLANT FORMULATIONS AND METHODS OF DELIVERY

(71) Applicant: James Chuang, Henderson, NV (US)

(72) Inventor: James Chuang, Henderson, NV (US)

(21) Appl. No.: 17/335,875

(22) Filed: Jun. 1, 2021

Related U.S. Application Data
(60) Provisional application No. 63/033,179, filed on Jun. 1, 2020.

(51) Int. Cl. A61K 36/534; A61K 36/752; A61K 31/05; A61K 31/4045; A61K 31/015; A61K 9/00

(52) U.S. Cl. CPC A61K 36/534; A61K 36/752; A61K 31/05; A61K 9/007; A61K 31/015; A61K 9/0014; A61K 9/0053; A61K 31/4045

(57) ABSTRACT

Disclosed are various embodiments of natural plant compositions. Particularly, the natural plant compositions may consist of plant chemicals, such as terpenes and cannabinoids, natural oils such as hemp seed oil and essential oils such as peppermint oil and lemon oil. Delivery methods for administration of the compositions include topical administration, an oral administration and a pulmonary administration. The compositions are used for a variety of reasons ranging from medicinal to recreational.

### CROSS-REFERENCE TO RELATED APPLICATION

The present application is related to and claims priority to U.S. Provisional Patent Application No. 63/033,179 filed Jun. 1, 2020, which is incorporated by reference herein in its entirety.

### BACKGROUND OF THE INVENTION

The following includes information that may be useful in understanding the present disclosure. It is not an admission that any of the information provided herein is prior art nor material to the presently described or claimed inventions, nor that any publication or document that is specifically or implicitly referenced is prior art.

### 1. FIELD OF THE INVENTION

The present invention relates generally to the field of plant formulations and more specifically relates to compositions formulated from plants, particularly cannabis plants, and methods of delivery of the same.

### 2. DESCRIPTION OF RELATED ART

Cannabinoids and terpenes are chemicals found in the cannabis plant. With the legalization of cannabis in many areas of the world, there has been increasing studies into the effects of cannabinoids and terpenes. One example of a cannabinoid is Cannabidiol (CBD). Research has found that CBD may have multiple medicinal, therapeutic, and recreational applications. For example, CBD may be used for the treatment of epilepsy, inflammation, chronic pain, asthma, mood and anxiety disorders, sleep disorders, eating disorders, etc. with very few side effects when using a proper dosage.

Terpenes are responsible for the unique aroma of each cannabis plant as well as the plant's regeneration, oxygenation, and immunity defense. Research has found that terpenes may support other cannabis chemicals (such as cannabinoids) in producing desired effects, in what is called an “entourage effect”, thus bolstering the health benefits associated therewith.

Due to the important health benefits that cannabinoids and terpenes can bring, it is desirable to provide as an effective dose as possible. However, current formulations are not satisfactory due to low bioavailability, an ineffective combination, failure to deliver one formulation that can be administered using different methods, etc. Further, current delivery methods are not suitable as they utilize high heat, causing potentially dangerous and counter-productive health side effects. Therefore, a suitable solution is desired.

U.S. Pat. No. 10,525,093 to Kenton L. Crowley relates to cannabinoid formulations and method of making the same. The described cannabinoid formulations and method of making the same includes a composition comprising cannabis extract and melatonin is provided, along with formulations for delivering the composition to a subject. The composition can include a base ingredient such as polyethylene glycol, gelatin, tapioca and/or pectin; natural sweeteners; and oils of peppermint, lavender, ginger, citrus, mango, etc. The formulation can include tablets, capsules, lozenges, troches, suppositories, tinctures, a transdermal patch, a vaporizer, a metered dose inhaler, etc. The formulation avoids harmful preservatives (e.g., BHT, BHA), heavy metals and stabilizers while addressing sub-therapeutic dosing and optimizing the synergistic qualities of the ratios of the individual cannabinoids (both acidic and neutral forms), terpenes and flavonoids.

### BRIEF SUMMARY OF THE INVENTION

In view of the foregoing disadvantages inherent in the known plant formulations art, the present disclosure provides novel cannabis plant formulations and methods of delivery therefor. The general purpose of the present disclosure, which will be described subsequently in greater detail, is to provide various compositions utilizing plant chemicals, particularly cannabis plant chemicals such as cannabinoids and terpenes, and oils.

Various compositions comprising cannabis plant chemicals are disclosed herein. In some embodiments, cannabinoid only compositions may be provided. The cannabinoid only compositions may comprise Cannabis sativa hemp seed oil, at least one cannabinoid, and at least one essential oil. Further, some embodiments of the cannabinoid compositions may comprise melatonin. The at least one cannabinoid may be cannabidiol. The at least one essential oil may include at least one of peppermint oil and/or lemon oil, providing a pleasant taste and/or aroma to the compositions.

In some embodiments, terpene only compositions may be provided. The terpene only compositions may comprise Cannabis sativa hemp seed oil, at least one terpene, and at least one essential. The at least one terpene may include at least one of beta-caryophyllene, myrcene, limonene, alpha-pinene, and/or linalool. The at least one essential oil may peppermint oil, again providing a pleasant taste and/or aroma to the compositions. In further embodiments, combination terpene and cannabinoid compositions may be provided. The combination compositions may comprise Cannabis sativa hemp seed oil, at least one cannabinoid, at least one terpene and at least one essential oil.

For purposes of summarizing the invention, certain aspects, advantages, and novel features of the invention have been described herein. It is to be understood that not necessarily all such advantages may be achieved in accordance with any one particular embodiment of the invention. Thus, the invention may be embodied or carried out in a manner that achieves or optimizes one advantage or group of advantages as taught herein without necessarily achieving other advantages as may be taught or suggested herein. The features of the invention which are believed to be novel are particularly pointed out and distinctly claimed in the concluding portion of the specification. These and other features, aspects, and advantages of the present invention will become better understood with reference to the following drawings and detailed description.

### BRIEF DESCRIPTION OF THE DRAWINGS

The figures which accompany the written portion of this specification illustrate embodiments and methods of use for the present disclosure, cannabis plant formulations, constructed and operative according to the teachings of the present disclosure.

FIG. 1 is a table illustrating a cannabinoid only composition according to an embodiment of the disclosure.

FIG. 2 is a table illustrating a cannabinoid only composition according to another embodiment of the disclosure.

FIG. 3 is a table illustrating a terpene only composition according to an embodiment of the disclosure.

FIG. 4 is a table illustrating a terpene only composition according to another embodiment of the disclosure.

FIG. 5 is a table illustrating a terpene only composition according to another embodiment of the disclosure.

FIG. 6 is a table illustrating a combination cannabinoid and terpene composition according to an embodiment of the disclosure.

FIG. 7 is a table illustrating a combination cannabinoid and terpene composition according to another embodiment of the disclosure.

FIG. 8 is a table illustrating a combination cannabinoid and terpene composition according to another embodiment of the disclosure.

The various embodiments of the present invention will hereinafter be described in conjunction with the appended drawings, wherein like designations denote like elements.

### DETAILED DESCRIPTION

As discussed above, embodiments of the present disclosure relate to therapeutic formulations and more particularly to natural plant compositions. Specifically, the natural plant compositions may be formulated from plant chemicals, such as terpenes and cannabinoids, natural oils such as hemp seed oil and essential oils such as peppermint oil and lemon oil.

Generally disclosed are various compositions having a variety of uses. Particularly, the compositions may be used as an analgesic, a bronchodilator, an anticonvulsant, anti-hyperalgesic, an antibacterial, an anti-fungal, an antioxidant, etc. The various compositions may also be used for the treatment of a variety of conditions. For example, the anti-inflammatory and antispasmodic properties of the compositions may reduce and prevent asthma attacks. Some other conditions the compositions may be used to treat may be addiction, anxiety, depression, asthma, epilepsy, Parkinson's disease, rash, inflammation, eczema, psoriasis, Post Traumatic Stress Disorder, etc.

In addition to this, the various compositions may be used to treat infection and disease from certain viruses. For example, the anti-inflammatory, anti-spasmodic and cell-regenerative properties of the compositions may be used to treat certain complications from COVID-19 (or other strains thereof). For instance, the compositions may prevent serious respiratory disorders associated with COVID-19, especially for the asthmatic, the elderly or those with preexisting conditions. In another example, the compositions may treat complications such as Multisystem Inflammatory Syndrome for Children (MIS-C).

The various compositions may essentially consist of natural food grade plant chemicals and oils. Particularly, the plant chemicals may comprise of at least one cannabinoid and/or at least one terpene which may preferably be derived from the cannabis plant. The various compositions may preferably be administered to a user via one of three delivery methods, which will be discussed in further detail below.

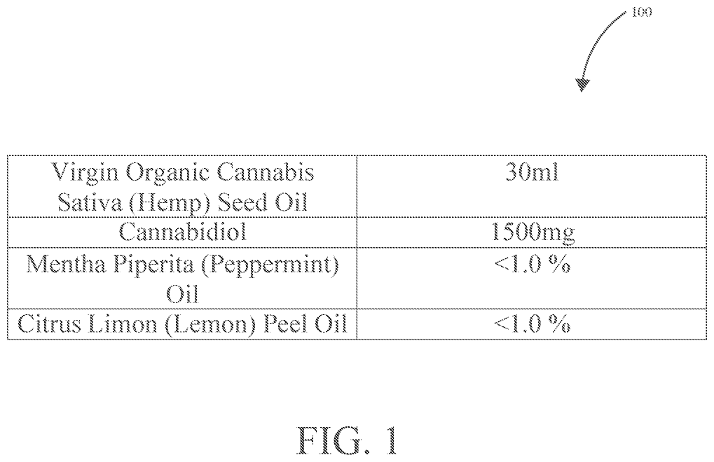

Referring now more specifically to the drawings by numerals of reference, there is shown in FIGS. 1-8, various tables depicting the various compositions, and methods of delivery thereof disclosed thereafter.

In some embodiments, cannabinoid only compositions may be provided. The cannabinoid only compositions may comprise Cannabis sativa hemp seed oil, at least one cannabinoid, and at least one essential oil. Further, some embodiments of the cannabinoid compositions may comprise melatonin. The at least one cannabinoid may be cannabidiol. The at least one essential oil may include at least one of peppermint oil and/or lemon oil.

FIG. 1 shows a composition 100 according to one embodiment of a cannabinoid only composition. As shown in this figure, the composition 100 may comprise Cannabis sativa hemp seed oil, cannabidiol, peppermint oil and lemon oil. As shown, a quantity of the Cannabis sativa hemp seed oil may be 30 ml. This composition 100 may comprise cannabinoid isolates or may include full-spectrum cannabinoids. In the full-spectrum CBD example, the composition may further comprise tetrahydrocannabinol (THC).

The combination of Cannabis sativa hemp seed oil and the cannabidiol may create an “entourage effect”, further increasing bioavailability of the cannabidiol (and/or any other cannabinoid included in the composition). As such, the cannabidiol may be provided in the composition in larger effective amounts. For example, as shown in FIG. 1, a quantity of the cannabidiol may be 1500 mg.

As above and as shown in FIG. 1, the composition 100 may comprise both peppermint oil and lemon oil. A quantity of the peppermint oil may be less than 1.0% of a total weight of the composition 100, and likewise, a quantity of the lemon oil may be less than 1.0% of the total weight of the composition 100. The peppermint oil and the lemon oil may be used to provide a pleasant taste and/or aroma to the composition 100, as without the essential oils, the composition may be substantially scent-less and tasteless. It should be appreciated however that other essential oils may be added (in addition to or instead of the peppermint oil and/or the lemon oil). For example, the essential oil may be chosen and added by the user.

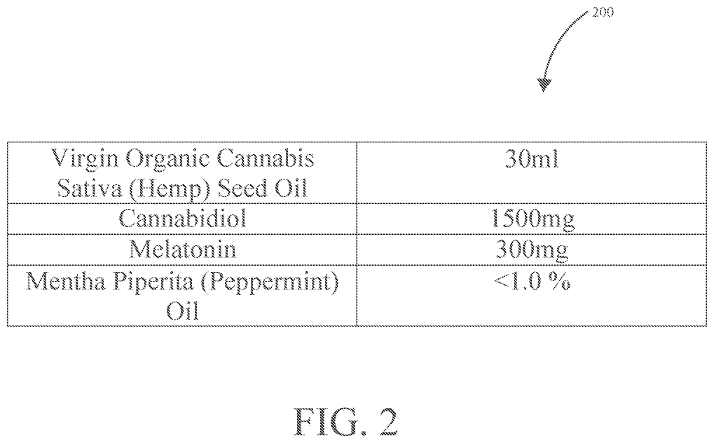

Referring specifically to FIG. 2, there is shown a composition 200 according to another embodiment of a cannabinoid only composition. As shown in this figure, the composition 200 may comprise Cannabis sativa hemp seed oil, cannabidiol, peppermint oil and melatonin. This composition 200 may also comprise cannabinoid isolates or may include full-spectrum cannabinoids, and again, in the full-spectrum CBD example, the composition 200 may further comprise tetrahydrocannabinol (THC).

Similarly, to the composition 100 above, a quantity of the Cannabis sativa hemp seed oil may be 30 ml, a quantity of the cannabidiol may be 1500 mg, and a quantity of the peppermint oil may be less than 1.0% of a total weight of the composition 200. A quantity of the melatonin may be 300 mg. This composition 200 may preferably be used as a sleep aid. Other forms of sleep-inducing chemicals may also be contemplated for addition to the composition 200. For example, valerian root, lavender essential oil, etc. Again, it should be appreciated that the essential oils may be chosen and added by the user and this composition 200 is not limited to peppermint oil.

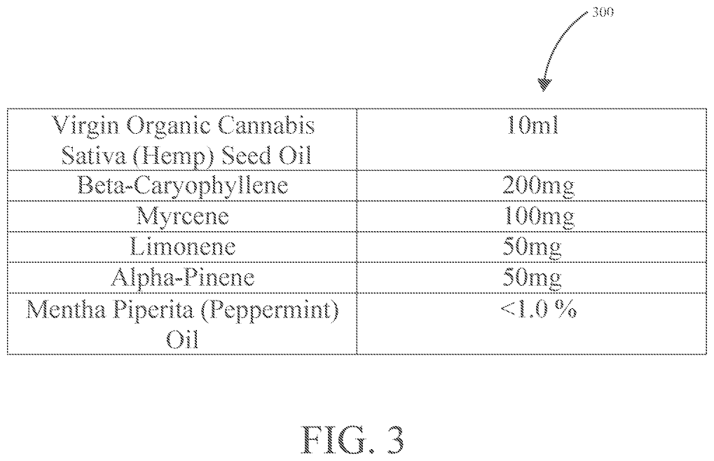

Referring now to FIGS. 3-5, in some embodiments, terpene only compositions may be provided. The terpene only compositions may comprise Cannabis sativa hemp seed oil, at least one terpene, and at least one essential. FIG. 3 shows a composition 300 according to one embodiment of a terpene only composition. As shown, the composition 300 may comprise Cannabis sativa hemp seed oil, beta-caryophyllene, myrcene, limonene, alpha-pinene and peppermint oil.

As shown in FIG. 3, a quantity of Cannabis sativa hemp seed oil may be 10 ml, a quantity of the beta-caryophyllene may be 200 mg, a quantity of myrcene may be 100 mg, a quantity of limonene may be 50 mg, a quantity of alpha-pinene may be 50 mg, and a quantity of the peppermint oil may be less than 1.0% of a total weight of the composition 300. Again, the composition may not be limited to the peppermint oil.

In some embodiments, the composition 300 may be a sativa composition (taken from a Cannabis sativa plant). In some embodiments, the terpene only compositions may be used with the cannabinoid only compositions, creating a desired “entourage effect” for the user. Further, in some embodiments, the composition 300 may be applied into foods and beverages.

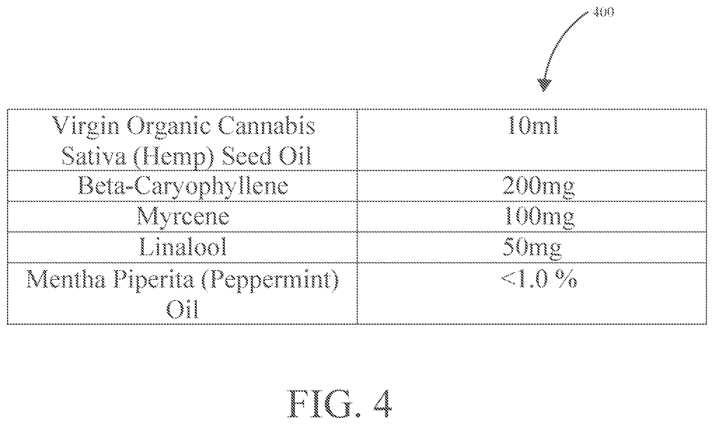

Referring specifically to FIG. 4, there is shown a composition 400 according to another embodiment of a terpene only composition. As shown, the composition 400 may comprise Cannabis sativa hemp seed oil, beta-caryophyllene, myrcene, linalool and peppermint oil. This composition 400 may be an indica composition (taken from a cannabis indicia plant). A quantity of the Cannabis sativa hemp seed oil may be 10 ml, a quantity of the beta-caryophyllene may be 200 mg, a quantity of the myrcene may be 100 mg, a quantity of the linalool may be 50 mg and a quantity of the peppermint oil may be less than 1.0% of a total weight of the composition 400. Again, the composition 400 may not be limited to the peppermint oil.

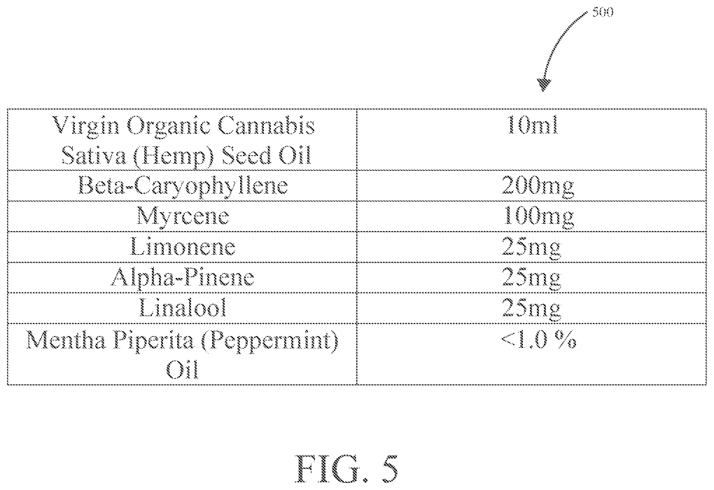

Referring specifically to FIG. 5, there is shown a composition 500 according to another embodiment of a terpene only composition. As shown, the composition 500 may comprise Cannabis sativa hemp seed oil, beta-caryophyllene, myrcene, limonene, alpha-pinene, linalool, and peppermint oil. This composition 500 may be a hybrid composition (taken from a hybrid cannabis plant being cross-bred from sativa and indica plants). A quantity of the Cannabis sativa hemp seed oil may be 10 ml, a quantity of the beta-caryophyllene may be 200 mg, a quantity of the myrcene may be 100 mg, a quantity of the limonene may be 25 mg, a quantity of the alpha-pinene may be 25 mg, a quantity of the linalool may be 25 mg and a quantity of the peppermint oil may be less than 0.1% of a total weight of the composition 500. Again, the composition 500 may not be limited to the peppermint oil.

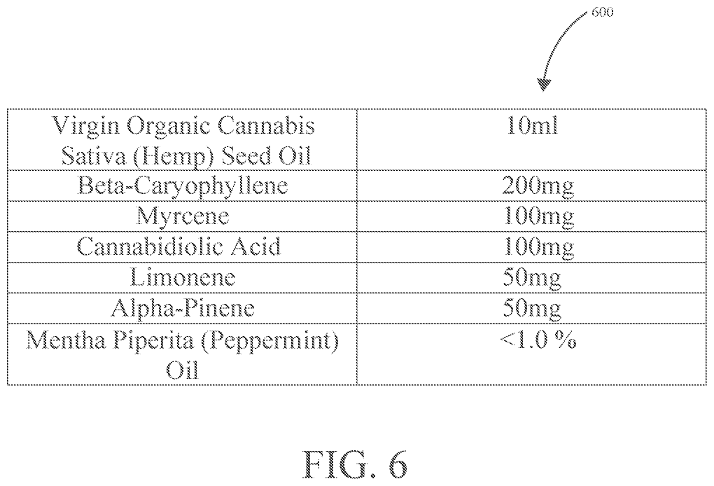

Referring to FIGS. 6-8, as shown, in some embodiments, combination terpene and cannabinoid compositions may be provided. In some embodiments, the combination terpene and cannabinoid compositions may comprise ingredients similar to the terpene only compositions discussed above with the addition of at least one cannabinoid. For example, FIG. 6 shows a composition 600 according to one embodiment of a combination terpene and cannabinoid composition. As shown, the composition 600 may comprise Cannabis sativa hemp seed oil, beta-caryophyllene, myrcene, limonene, alpha-pinene, peppermint oil and cannabidiolic acid (CBDA). The inclusion of CBDA in the combination may activate CB1 and CB2 receptors in the user. Combining the composition with CBD may produce longer and stronger effects. Whilst the CBDA is a preferred embodiment, the composition 600 may also include CBD as well as, or instead of, the CBDA.

As shown in FIG. 6, a quantity of Cannabis sativa hemp seed oil may be 10 ml, a quantity of the beta-caryophyllene may be 200 mg, a quantity of myrcene may be 100 mg, a quantity of limonene may be 50 mg, a quantity of alpha-pinene may be 50 mg, a quantity of the cannabidiolic acid may be 100 mg, and a quantity of the peppermint oil may be less than 1.0% of a total weight of the composition 600. Again, the composition 600 may not be limited to the peppermint oil.

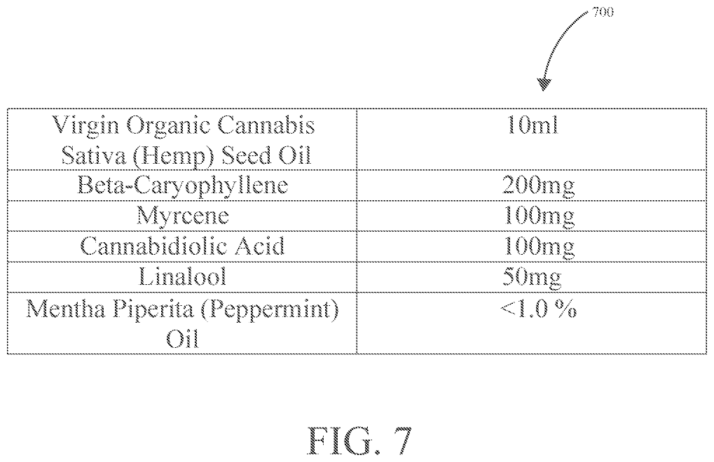

Referring now to FIG. 7 showing a composition 700 according to another embodiment of a combination terpene and cannabinoid composition. As shown, the composition 700 may comprise Cannabis sativa hemp seed oil, beta-caryophyllene, myrcene, linalool and peppermint oil. This composition 700 may be an indica composition (taken from a cannabis indica plant). A quantity of the Cannabis sativa hemp seed oil may be 10 ml, a quantity of the beta-caryophyllene may be 200 mg, a quantity of the myrcene may be 100 mg, a quantity of the linalool may be 50 mg, a quantity of the cannabidiolic acid may be 100 mg, and a quantity of the peppermint oil may be less than 1.0% of a total weight of the composition 700. Again, the composition 700 may not be limited to the peppermint oil.

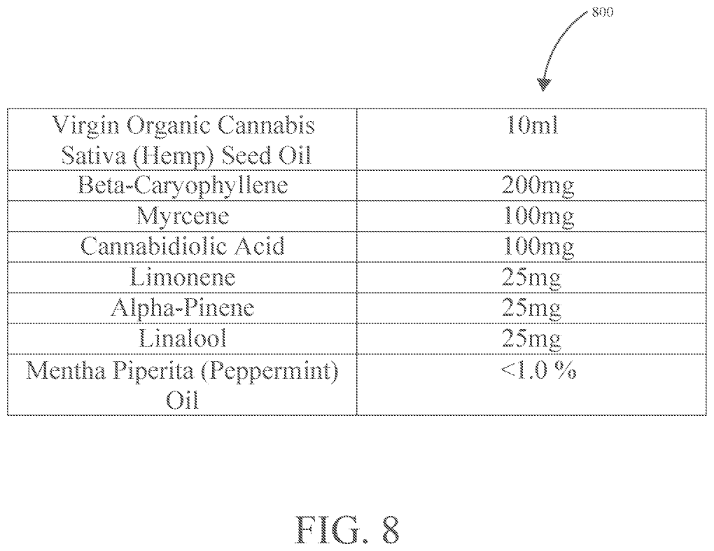

Referring now to FIG. 8 showing a composition 800 according to another embodiment of a combination terpene and cannabinoid composition. As shown, the composition 800 may comprise Cannabis sativa hemp seed oil, beta-caryophyllene, myrcene, limonene, alpha-pinene, linalool, and peppermint oil. This composition 800 may be a hybrid composition (taken from a hybrid cannabis plant being cross-bred from sativa and indica plants). A quantity of the Cannabis sativa hemp seed oil may be 10 ml, a quantity of the beta-caryophyllene may be 200 mg, a quantity of the myrcene may be 100 mg, a quantity of the limonene may be 25 mg, a quantity of the alpha-pinene may be 25 mg, a quantity of the linalool may be 25 mg, a quantity of the cannabidiolic acid may be 100 mg, and a quantity of the peppermint oil may be less than 0.1% of a total weight of the composition 800. Again, the composition 800 may not be limited to the peppermint oil.

It should be appreciated that whilst cannabidiol (CBD) and cannabidiolic acid have been given as the preferred components of the compositions, any cannabinoid and/or any active chemical in cannabis may be added to any of the compositions. For example, cannabidivarin (CBDV), cannabigerol (CBG), THC, tetrahydrocannabivarin (THCV), cannabinol (CBN), etc.

It is contemplated that all above discussed compositions may be formulated for safe and effective delivery to a user by at least one of a topical administration, an oral administration and a pulmonary administration. Preferably, the safe and effective delivery may utilize little to no heat, thus preventing any dangerous side effects caused by heating cannabis chemicals (such as CBD and terpenes) and dangerous side effects involved in inhaling heated vapor whilst also preventing loss of potency involved with heating the components.

Three different delivery methods are disclosed for the administration of any of the above compositions. Preferably all above discussed compositions may be formulated as an oil. The oil may be provided in a bottle, and in some embodiments, a pipette or other form of dosage-providing means may be included, enabling the user to utilize a desired dosage of the compositions. The oil may allow a user is able to perform any of the delivery methods using any of the compositions. For example, the user may utilize the topical administration with composition one day, and oral administration with composition another day.

The first delivery method disclosed herein is a topical delivery method. The topical delivery method may include the steps of: providing the composition (any of the above discussed compositions); applying (at least one) dose of the composition onto a body surface; and massaging the dose of the composition into the body surface. The body surface may be skin of the user. In some examples, the user may apply 1-2 drops of any of the compositions (depending on size of the body part) and massage into the skin. The compositions may preferably be non-comedogenic and high in antioxidants so as to not clog the skin.

The second delivery method disclosed is an oral delivery method. The oral delivery method may include the steps of: providing the composition (any of the above discussed compositions); introducing (at least one) dose of the composition into an oral cavity of the user; and swallowing the dose or applying the dose under a tongue of the user (for sublingual administration). Sublingual administration may increase bioavailability of the compositions whilst decreasing activation time. The oral delivery method may be used for administering the composition(s) as a dietary supplement.

The third delivery method disclosed is a pulmonary delivery method. The pulmonary delivery method may include the steps of: providing the composition (any of the above discussed compositions); adding (at least one) dose of the composition into an aero-solution generating device, the aero-solution generating device turning the dose substantially into an aero-solution; and inhaling the aero-solution. The aero-solution generating device may be an existing aromatherapy diffuser, humidifier, mister, nebulizer, etc. Further, in some embodiments, the composition may be added to central air systems for delivery through air vents in a building. In some embodiments, fragrant oils may be added with the composition to provide aromatherapy without interfering with the benefits of the compositions.

It should be noted that the steps described in the method of use can be carried out in many different orders according to user preference. The use of “step of” should not be interpreted as “step for”, in the claims herein and is not intended to invoke the provisions of 35 U.S.C. § 112(f). It should also be noted that, under appropriate circumstances, considering such issues as design preference, user preferences, marketing preferences, cost, structural requirements, available materials, technological advances, etc., other methods for the administration of natural plant formulations (e.g., different step orders within above-mentioned list, elimination, or addition of certain steps, including or excluding certain maintenance steps, etc.), are taught herein.

The embodiments of the invention described herein are exemplary and numerous modifications, variations and rearrangements can be readily envisioned to achieve substantially equivalent results, all of which are intended to be embraced within the spirit and scope of the invention. Further, the purpose of the foregoing abstract is to enable the U.S. Patent and Trademark Office and the public generally, and especially the scientist, engineers and practitioners in the art who are not familiar with patent or legal terms or phraseology, to determine quickly from a cursory inspection the nature and essence of the technical disclosure of the application.

What is claimed is new and desired to be protected by Letters Patent is set forth in the appended claims:

1. A composition consisting essentially of:
Cannabis sativa hemp seed oil;
cannabidiol;
peppermint oil;
lemon oil; and
wherein the composition is formulated for safe and effective delivery to a user by at least one of a topical administration, an oral administration and a pulmonary administration.

2. The composition of claim 1, wherein a quantity of Cannabis sativa hemp seed oil is about 30 ml.

3. The composition of claim 1, wherein a quantity of cannabidiol is about 1500 mg.

4. The composition of claim 1, wherein a quantity of peppermint oil is about less than 1.0% of a total weight of the composition.

5. The composition of claim 1, wherein a quantity of lemon oil is about less than 1.0% of the total weight of the composition.

6. A composition consisting of:
Cannabis sativa hemp seed oil;
cannabidiol;
melatonin;
peppermint oil; and
wherein the composition is formulated for safe and effective delivery to a user by at least one of a topical administration, an oral administration and a pulmonary administration.

7. The composition of claim 6, wherein a quantity of Cannabis sativa hemp seed oil is about 30 ml.

8. The composition of claim 6, wherein a quantity of cannabidiol is about 1500 mg.

9. The composition of claim 6, wherein a quantity of melatonin is about 300 mg.

10. The composition of claim 6, wherein a quantity of peppermint oil is about less than 1.0% of a total weight of the composition.

11. A composition consisting of:
Cannabis sativa hemp seed oil;
beta-caryophyllene;
myrcene;
limonene;
alpha-pinene;
peppermint oil; and
wherein the composition is formulated for safe and effective delivery to a user by at least one of a topical administration, an oral administration and a pulmonary administration.

12. The composition of claim 11, wherein a quantity of Cannabis sativa hemp seed oil is about 10 ml.

13. The composition of claim 11, wherein a quantity of beta-caryophyllene is about 200 mg.

14. The composition of claim 11, wherein a quantity of myrcene is about 100 mg.

15. The composition of claim 11, wherein a quantity of limonene is about 50 mg.

16. The composition of claim 11, wherein a quantity of alpha-pinene is about 50 mg.

17. The composition of claim 11, wherein a quantity of peppermint oil is about less than 1.0% of a total weight of the composition.

18. The composition of claim 11, wherein the composition further consists of cannabidiolic acid.

19. The composition of claim 18, wherein a quantity of cannabidiolic acid is about 100 mg.

20. A composition consisting of:
Cannabis sativa hemp seed oil;
beta-caryophyllene;
myrcene;
linalool;
peppermint oil; and
wherein the composition is formulated for safe and effective delivery to a user by at least one of a topical administration, an oral administration and a pulmonary administration.

21. The composition of claim 20, wherein a quantity of Cannabis sativa hemp seed oil is about 10 ml.

22. The composition of claim 20, wherein a quantity of beta-caryophyllene is about 200 mg.

23. The composition of claim 20, wherein a quantity of myrcene is about 100 mg.

24. The composition of claim 20, wherein a quantity of linalool is about 50 mg.

25. The composition of claim 20, wherein a quantity of peppermint oil is about less than 1.0% of a total weight of the composition.

26. The composition of claim 20, wherein the composition further consists of cannabidiolic acid.

27. The composition of claim 26, wherein a quantity of cannabidiolic acid is about 100 mg.

28. A composition consisting of:
Cannabis sativa hemp seed oil;
beta-caryophyllene;
myrcene;
limonene;
alpha-pinene;
linalool;
peppermint oil; and
wherein the composition is formulated for safe and effective delivery to a user by at least one of a topical administration, an oral administration and a pulmonary administration.

29. The composition of claim 28, wherein a quantity of Cannabis sativa hemp seed oil is about 10 ml.

30. The composition of claim 28, wherein a quantity of beta-caryophyllene is about 200 mg.

31. The composition of claim 28, wherein a quantity of myrcene is about 100 mg.

32. The composition of claim 28, wherein a quantity of limonene is about 25 mg.

33. The composition of claim 28, wherein a quantity of alpha-pinene is about 25 mg.

34. The composition of claim 28, wherein a quantity of linalool is about 25 mg.

35. The composition of claim 28, wherein a quantity of peppermint oil is about less than 1.0% of a total weight of the composition.

36. The composition of claim 28, wherein the composition further consists of cannabidiolic acid.

37. The composition of claim 36, wherein a quantity of cannabidiolic acid is about 100 mg.
